(19) United States
(12) Patent Application Publication
(10) Pub. No.: US 2006/0292681 A1
(43) Pub. Date: Dec. 28, 2006

(54) Method for designing modulators of flexible multi-domain protein kinases

(75) Inventor: Thomas K. Harris, Miami, FL (US)

Correspondence Address: WILMER CUTLER PICKERING HALE AND DORR LLP, 1875 PENNSYLVANIA AVE., NW, WASHINGTON, DC, 20004, US

(73) Assignee: University of Miami, Miami, FL (US)

(21) Appl. No.: 11/450,295

(22) Filed: Jun. 12, 2006

Related U.S. Application Data
(60) Provisional application No. 60/689,533, filed on Jun. 13, 2005.

(51) Int. Cl. C12N 9/99; G06F 19/00

(52) U.S. Cl. 435/184; 702/19

(57) ABSTRACT

The present invention describes novel methods of designing protein kinase modulators, particularly modulators that interact at the interface between two protein kinase domains. In some embodiments, the methods comprise determining the position of at least one site between the first domain and the second domain, wherein the modulator binds to the at least one site and wherein the at least one site is not an ATP binding site.

### RELATED APPLICATIONS

This application claims the benefit under 35 U.S.C. § 119(e) of U.S. Provisional Application No. 60/689,533, filed on Jun. 13, 2005, which is hereby incorporated by reference herein in its entirety.

### GOVERNMENT SUPPORT

Work described herein may have been supported in part by NIH Grant number GM069868. The U.S. Government may have certain rights in the invention.

### FIELD OF THE INVENTION

The present invention is directed generally to the field of molecular biology. More particularly, the present invention describes novel methods of designing protein kinase modulators, particularly modulators that interact at the interface between two protein kinase domains.

### BACKGROUND OF THE INVENTION

Primary cancer treatment currently involves surgical removal of malignant tumors, followed by radiation and/or chemotherapy. The majority of FDA-approved chemotherapeutic agents, such as the alkylating agents, natural products, and antimetabolites that are used in combination treatment regimens, target rapidly dividing cells. While such agents effectively kill proliferating tumor cells, they also kill normal dividing cells, which results in their high toxicity.

More effective treatments are beginning to emerge from the use of targeted therapies such as the FDA-approved drugs Gleevec™ (Novartis), Iressa™ (AstraZeneca), Tarceva™ (Roche/Genentech). These new forms of targeted therapies bind and inactivate specific “oncogenic” cell cycle control factors (i.e., protein kinases) so that the ratio of damage incurred by normal cells over tumor cells is dramatically decreased and side effects become much less severe. A variety of human oncogenic protein kinases have been identified for development of targeted therapies, and they account for 20-30% of the drug discovery programs in the pharmaceutical industry (1).

Despite the large amount of effort that has been directed towards the development of modulators of protein kinases (2), only a few compounds have been FDA approved for clinical use. The primary reason that so few protein kinase modulators have been approved for clinical use is that an overwhelming majority of compounds that inhibit a targeted protein kinase mimic ATP and bind in the ATP binding pocket causing inhibition of numerous other kinases and ATPases (3,4). Although Gleevec™ has been shown to be competitive with ATP, X-ray diffraction studies indicate that the relatively high affinity and specificity is due to an additional hydrophobic moiety, which extends into a hydrophobic pocket near the ATP-binding site of only the inactive form of the kinase (5). The extended hydrophobic moiety exemplifies the additive nature of binding free energies of chemically linked molecular fragments (6). Thus, additive binding can facilitate the search for drugs in fragments, which offers a tremendous combinatorial advantage over discovery of drugs intact. However, the primary challenge of detecting weak interactions of small molecular fragments with proteins continues to hinder fragment-based drug design and development.

One of the most recent and promising methods of fragment-based drug discovery entails Tethering™ (7). The basic method of tethering is to engineer a single-site surface-exposed cysteine residue within 5 to 10 Å of a potential binding pocket of interest. Then, the protein is reacted with a library of disulfide containing fragments under partially reducing conditions. If one of the fragments has inherent affinity to a site near the cysteine, the thiol-disulfide equilibrium will be shifted in favor of the disulfide for this fragment. Due to the change in mass by the amount of the particular disulfide-linked fragment, the predominant chemically modified protein species can be identified by mass spectrometry. Then selected fragments can be elaborated, combined with other molecules, or combined with one another to provide high-affinity drug leads. The primary advantage of Tethering™ is that it provides a site-directed basis for fragment-based drug discovery based on low to moderate binding activities in contrast to selection based on high-affinity inhibition of enzyme activity by intact drugs required of high throughput screening assays. One major limitation to the tethering approach, as well as for other structure-based drug discovery methods such as X-ray crystallography and NMR, is the feasibility of (i) obtaining three-dimensional models of all target proteins and (ii) detecting additional small molecule binding sites in flexible loop regions that may exist some distance from the active site.

Structure-based approaches represent the newest and most promising areas of focus for drug discovery programs (8). Numerous companies have begun to automate the structural biology process to rapidly crystallize gene products on a massive scale (e.g., Astex, Structural Genomix, and Syrrx). However, it is becoming evident that X-ray diffraction quality crystals are difficult to obtain for many of the most important protein targets, since they behave as flexible multi-domain proteins (9). In such cases, X-ray or NMR structures can be solved only for individual functional domains. For example, FIG. 1 shows domain organizations of number of well-characterized serine-threonine protein kinases of pharmaceutical interest. Of these members, X-ray structures have been reported only for isolated kinase domain constructs of phosphoinositide-dependent protein kinase-1 (PDK1), protein kinase B/AKT (PKB), protein kinase A (PKA), c-JUN NH₂-terminal protein and 38-kDA protein kinases (JNK/p38), extracellular signal-regulated protein kinases (ERK1,2), cyclin-dependent protein kinases (CDK), and checkpoint kinases (CHK); and X-ray or NMR structures have been reported only for the corresponding isolated regulatory domain constructs of the pleckstrin homology (PH) domain of PKB, the diacylglycerol (DAG) and Ca²⁺ domains of PKC, the cyclic adenosine monophosphate (cAMP)-regulatory subunit of PKA, the cyclin-regulatory subunit of CDK, the conserved region 1 (CR1) domain of the first identified downstream effector kinases (RAF1,A,B), and the forkhead-associated (FHA) domain of CHK.

Knowledge of the structures of the full-length multi-domain protein kinases would uncover new tactical sites of interest to which small molecular fragments can be targeted for binding interactions. Also, fragment-based drug discovery methods such as Tethering™ could be better exploited if these structures could be determined. For example, chemically linked compounds could be developed that could simultaneously interact with either known binding pockets of proximal domains or unknown potential binding pockets that may exist within clefts or crevices near the interface between contiguous domains.

There is a need for a method of designing protein kinase modulators that target tactical binding sites that may exist some distance from the active site. There is also a need for a method of designing protein kinase modulators with high selectivity for single protein kinases. The invention is directed to these and other important ends.

### SUMMARY OF THE INVENTION

The present invention provides novel methods of designing a protein kinase modulator to interact at the interface between a first domain and a second domain of a protein kinase.

In some embodiments, the methods comprise determining the position of at least one site between the first domain and the second domain, wherein the modulator binds to the at least one site and wherein the at least one site is not an ATP binding site.

In some embodiments, the methods comprise the steps of site-directed spin labeling a first kinase domain; isotopically labeling a second kinase domain; ligating the first kinase domain and the second kinase domain; and determining the position at least one site between the site-directed spin-labeled first domain and the isotopically-labeled second domain, wherein the modulator binds to at the least one site between the first domain and the second domain and wherein the at least one site is not an ATP binding site.

In some embodiments, the methods comprise the steps of isotopically labeling a first kinase domain; site-directed spin labeling a second kinase domain; ligating the first kinase domain and the second kinase domain; and determining the position of at least one site between the site-directed spin-labeled first domain and the isotopically-labeled second domain, wherein the modulator binds to the at least one site between the first domain and the second domain and wherein the at least one site is not an ATP binding site.

In some embodiments, the methods comprise identifying at least one small molecule fragment that binds to at least one site between the first domain and the second domain, wherein the at least one site is not an ATP binding site.

In some embodiments, the methods comprise the steps of identifying a first small molecule fragment that binds to at least a first site between the first domain and the second domain; identifying a second small molecule fragment that binds to at least a second site between the first domain and the second domain; and chemically linking the two fragments, wherein the first site and second site are not ATP binding sites.

In one aspect of the invention, the protein kinase modulator is designed to stabilize an inactive form of the kinase. In a further aspect of the invention, the modulator is designed to stabilize an inactive form of the kinase in an allosteric manner.

In another aspect of the invention, the at least one site is at an interfacial cleft or crevice between the first domain and the second domain.

Protein kinases targeted by the methods of the invention include PDK1; PKB2; PKA; JNK/p38; ERK1,2; CDK; and CHK.

In another aspect of the invention, the first domain and the second domain are contiguous. In one embodiment, the first domain is a regulatory domain and the second domain is a catalytic domain and in another embodiment, the first domain is a catalytic domain and the second domain is a regulatory domain. In yet another embodiment, the regulatory domain is a PH domain.

In one or more embodiments of the invention, magnetic resonance energy transfer studies are used to determine the position of the at least one site between the first domain and the second domain. In one embodiment, the first domain is site-directed spin-labeled and the second domain is isotopically-labeled and in another embodiment, the first domain is isotopically-labeled and the second domain is site-directed spin-labeled. The first domain and the second domain may be chemically ligated. In a further embodiment, the position of the at least one site between the first domain and the second domain is determined by obtaining the distance between the spin label on the first domain to each of the backbone amide protons of the isotopically-labeled second domain.

In other embodiments, the position of at least two sites between the first domain and the second domain is determined and the modulator is designed to bind to the at least two sites.

In other embodiments, the modulator further binds to at least one site on the first domain, and in other embodiments, the modulator further binds to at least one site on the second domain.

In some aspects of the invention, the modulator comprises at least one small molecule fragment. The fragment may bind to the at least one site between the first domain and the second domain.

In some aspects of the invention, the modulator comprises at least two small molecule fragments. In one embodiment, each fragment may bind to at least one site between the first domain and the second domain. In another embodiment, at least one fragment binds to at least one site between the first domain and the second domain and at least one fragment binds to at least one site on the first domain. In yet another embodiment, at least one fragment binds to at least one site between the first domain and the second domain and at least one fragment binds to at least one site on the second domain. The small molecule fragments may be chemically linked or disulfide-linked.

The invention also provides methods of modulating a protein kinase using a protein kinase modulator designed to interact at the interface between a first domain and a second domain of a protein kinase, wherein the modulator binds to the at least one site between the first domain and the second domain and wherein the at least one site is not an ATP binding site.

Further provided by the invention are the protein kinase modulators produced by the methods of the invention.

### BRIEF DESCRIPTION OF THE FIGURES

FIG. 1 illustrates the domain organization of well-characterized serine-threonine protein kinases of pharmaceutical interest. The isolated kinase domain constructs for which X-ray three-dimensional structures have been reported are indicated in black: phosphoinositide-dependent protein kinase-1 (PDK1); protein kinase B/Akt (PKB); cyclic AMP-dependent protein kinases (PKA); c-JUN NH₂-terminal protein and 38 kDa protein kinases (JNK/p38); extracellular signal-regulated protein kinases (ERK1, 2); cyclin-dependent protein kinases (CDK); and checkpoint kinases (CHK). The active kinase domains of each family member must be phosphorylated, and the upstream protein kinase activator is indicated above each domain. No upstream activator has been clearly identified for phosphorylation of the C-terminal hydrophobic motif (HM) domains of PKB, SGK, p70 S6K, PKC, and cyclic AMP-dependent protein kinase (PKA). The regulatory domain constructs for which X-ray or NMR structures have been reported are indicated in gray: pleckstrin homology (PH) domain of PKB; diacylglycerol (DAG) and Ca²⁺ domains of PKC; cAMP-regulatory subunit of PKA; cyclin-regulatory subunit of CDK; the conserved region 1 (CR1) domain of the first identified downstream effector kinases (RAF1, A, B) of the mitogen-activated G protein (RAS); the polo box domain (PBD) of polo-like kinase (PLK) and the forkhead-associated (FHA) domain of CHK. Regulatory domains that bind either small-molecule second messengers or regulatory proteins are indicated.

FIG. 2 illustrates a strategy utilized to combine site-directed nitroxide spin labeling of the N-terminal kinase domain with uniform ¹⁵N-isotopic labeling of the C-terminal regulatory PH domain of PDK1. First, a His₆ tagged single-site cysteine mutant of the N-terminal kinase domain fused with a C-terminal Mxe GyrA intein is expressed and affinity purified from Sf9 insect cells. Thiolytic cleavage of the C-terminal Mxe GyrA intein with 2-mercaptoethanesulfonate (MESNA) generates a C-terminal thioester on the N-terminal kinase domain. The C-terminal regulatory PH domain is expressed as a fusion protein containing an N-terminal His₆ tag with a Factor Xa protease cleavage site and affinity purified from E. coli grown in minimal media supplemented with the desired NMR active isotopic label. Cleavage with Factor Xa generates an N-terminal cysteine (NT-Cys). As a result of the chemical reactivity between the C-terminal thioester and the NT-Cys, native peptide bond formation occurs upon mixing the N-terminal kinase domain with the C-terminal regulatory PH domain. Full-length PDK1 is affinity purified and the His₆ tag is cleaved to form segmental isotopic-labeled PDK1 for NMR structural and dynamical studies. In order to provide for site-directed spin labeling, the thioester derivative of the N-terminal kinase is first reacted with (1-oxyl-2,2,5,5-tetramethyl-3-pyrroline-3-methyl)methanesulfonate (MTSL, Toronto Research Chemicals Inc., Canada), which modifies the single-site cysteine with the nitroxide spin label through disulfide bond formation. NMR relaxation studies of the paramagnetic enhancement effect caused by insertion of the spin label yields distances between the unpaired electron of the spin label and the ¹⁵N-isotopic labeled backbone amide protons of the regulatory PH domain.

FIG. 3 illustrates a strategy utilized to combine uniform ¹⁵N-isotopic labeling of the N-terminal regulatory PH domain with site-directed nitroxide spin labeling of the C-terminal kinase domain of PKB2. First, the N-terminal regulatory PH domain is expressed as a fusion protein containing an N-terminal His₆ tag and a C-terminal Mxe GyrA intein and affinity purified from E. coli grown in minimal media supplemented with the desired NMR active isotopic label. Thiolytic cleavage of the C-terminal Mxe GyrA intein with 2-mercaptoethanesulfonate (MESNA) generates a C-terminal thioester on the N-terminal regulatory PH domain. The C-terminal kinase domain is expressed as a fusion protein containing an N-terminal His₆ tag with a Factor Xa protease cleavage site and affinity purified from Sf9 insect cells. Cleavage with Factor Xa generates an N-terminal cysteine (NT-Cys). As a result of the chemical reactivity between the C-terminal thioester and the NT-Cys, native peptide bond formation occurs upon mixing the two protein fragments. Full-length PKB2 is affinity purified and the His₆ tag is cleaved to form segmental isotopic-labeled PKB2 for NMR structural and dynamical studies. In order to provide for site-directed spin labeling of the C-terminal kinase domain, the NT-Cys is first protected with ninhydrin before chemical modification of the internal single-site cysteine with the MTSL nitroxide spin label. Upon deprotection of NT-Cys, the spin-labeled kinase domain is ligated to the thioester derivative of the N-terminal isotopic-labeled regulatory PH domain. NMR relaxation studies of the paramagnetic enhancement effect caused by insertion of the spin label yields distances between the unpaired electron of the spin label and the ¹⁵N-isotopic labeled backbone amide protons of the regulatory PH domain.

### DETAILED DESCRIPTION OF THE INVENTION

For the purposes of promoting an understanding of the principles of the invention, reference will now be made to specific embodiment and specific language will be used to describe the same. It will nevertheless be understood that no limitation of the scope of the invention is thereby intended, such alteration and further modifications of the invention, and such further applications of the principles of the invention as illustrated herein, being contemplated as would normally occur to one skilled in the art to which the invention relates.

All terms as used herein are defined according to the ordinary meanings they have acquired in the art. Such definitions can be found in any technical dictionary or reference known to the skilled artisan, such as the McGraw-Hill Dictionary of Scientific and Techinical Terms (McGraw-Hill, Inc.), Molecular Cloning: A Laboratory Manual (Cold Springs Harbor, N.Y.), and Remington's Pharmaceutical Sciences (Mack Publishing, PA). These references, along with those references and patents cited herein are hereby incorporated by reference in their entirety.

According to one or more embodiments, the present invention is directed to a novel method of designing protein kinase modulators. A “modulator” refers to a compound capable of modulating the expression of a protein kinase. For example, a protein kinase modulator may enhance protein kinase expression. A protein kinase modulator may also be an inhibitor of a protein kinase. In one aspect of the invention, a protein kinase modulator is an inhibitor that stabilizes an inactive form of the kinase. In a further aspect of the invention, a protein kinase modulator is an inhibitor that stabilizes an inactive form of the kinase in an allosteric manner. The term “allosteric” is well known in the art and refers to a change in the shape and activity of an enzyme that results from molecular binding with a modulatory compound at a site other than the active site. For example, an allosteric protein kinase modulator may stabilize an active form of the kinase by binding to a site other than an ATP binding site.

In accordance with the invention, protein kinase modulators are designed to interact at the interface between two domains of a protein kinase. The interface between two domains includes any site at or near a surface forming a common boundary between the two domains, including any clefts or crevices at the interface or leading to the interface. The interface may be between two contiguous domains of a protein kinase. In one or more embodiments of the invention, the interaction of a modulator in clefts and crevices formed at or near the interface between domains stabilizes an auto-inhibited, inactive form of the kinase, resulting in high selectivity of a modulator for a single protein kinase.

After the position of at least one site at the interface between two domains is determined, a protein kinase modulator may then be designed to bind to the at least one site. In another aspect of the invention, the positions of at least two sites at the interface between two domains is determined and a protein kinase modulator may then be designed to bind to the at least two sites. Protein kinase modulators designed to bind to at least one interfacial site and also to at least one site on the domains themselves are also within the scope of the invention.

Fragment-based methods known to the skilled artisan may be used to design the protein kinase modulator. For example, using the tethering method, one or more small molecule fragments that bind to one or more sites at the interface between the first domain and the second domain may be identified. If two or more small molecule fragments that bind to interfacial sites are identified, they may be then be chemically linked to one another or combined with other molecules to provide high-affinity protein kinase modulators. Additionally, one or more small molecule fragments that bind to one or more interfacial sites may be chemically linked with small molecule fragments that bind to one or more sites on the domains themselves.

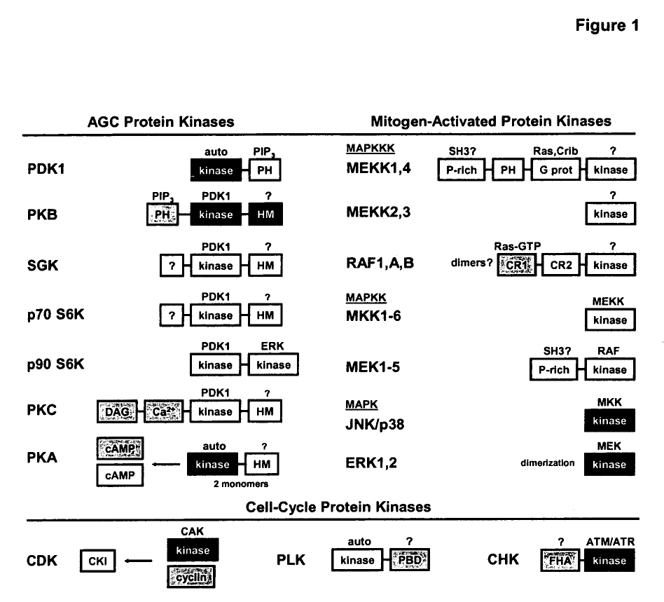

A novel structure-based approach is used to determine the position of at least one site at the interface between a first domain and a second domain of a protein kinase. This approach, named the “Magnetic Resonance Energy Transfer” (MRET) approach, combines the use of X-ray crystallography, segmental isotopic and site-directed spin labeling, and NMR structural studies and was developed by the inventors to study the structure and dynamics of flexible multi-domain protein kinase targets for which high quality three dimensional models for the full-length kinases have not able to be been determined. The invention can be used to design a highly selective modulator for any protein kinase, including, but not limited to, the protein kinases shown in FIG. 1.

Unpaired electrons of spin labels, such as the nitroxide spin label, produce local fluctuating magnetic fields, which affect the magnetic properties of the isotopic nuclei in a distance-dependent manner—the closer the amide is to the spin label, the more its relaxation rate is increased. This magnetic interaction is similar to the nuclear Overhauser effect (NOE) between pairs of protons, which is used for solving tertiary structures of proteins by identifying hydrophobic or van der waals contacts and hydrogen bonds. But unlike an NOE, whose measurable effect is limited to small distances ≦5.5 Å, the electron proton-interaction is much stronger and can extend up to 30 Å. In more general terms, the magnetic resonance energy transfer or MRET between the unpaired electron of a site-directed spin label and a proton in the same molecule is analogous to fluorescence resonance energy transfer (FRET) between donor and acceptor fluorophores. Unlike FRET determinations of the distance between one pair of fluorophore labels, MRET can be used to tremendous advantage in that distances can be obtained simultaneously between the site-directed spin label on a first domain and the backbone amide proton of all amino acid residues of the isotopic-labeled second domain.

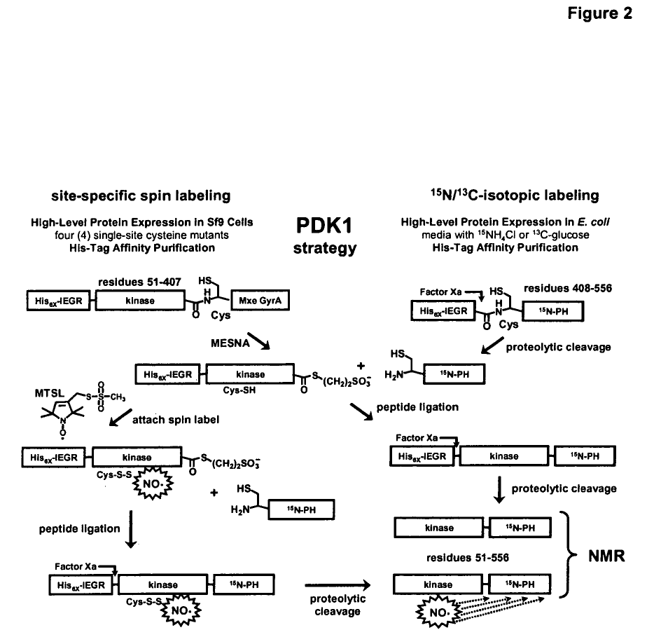

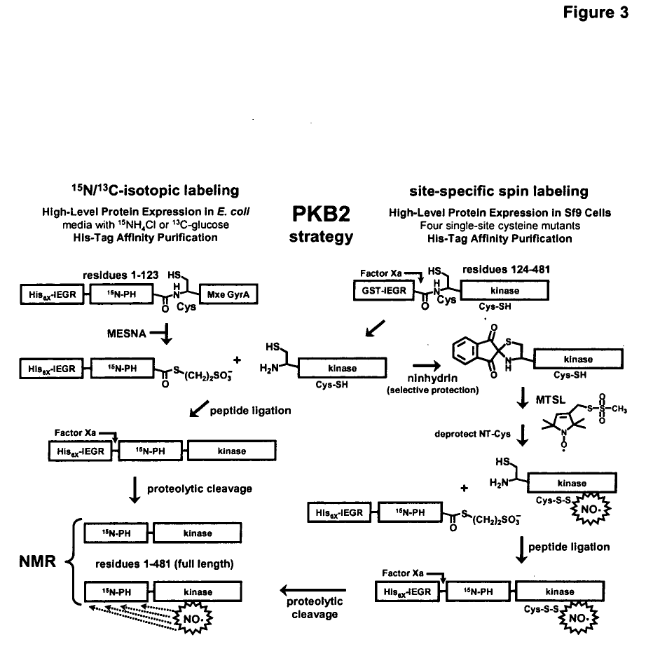

The first step of the MRET approach utilizes a combination of segmental isotopic labeling of one domain with site directed paramagnetic nitroxide spin labeling of a second domain to engineer protein kinase targets. In one or more embodiments of the invention, a regulatory domain, such as the pleckstrin homology (PH) domain, is selected for segmental isotopic labeling and a catalytic kinase domain is selected for site directed spin labeling. Since a regulatory domain may be positioned either amino (N)- or carboxy (C)-terminal to the kinase domain, two different protein engineering strategies were developed (FIGS. 2 and 3).

The serine-threonine protein kinases PDK1 and PKB2 provide model examples of the two different engineering strategies. The regulatory PH domain of PDK1 is located at the C-terminal end of its kinase domain, while the regulatory PH domain of PKB2 is located at the N-terminal end of its kinase domain. FIG. 2 illustrates site-directed spin labeling of the N-terminal kinase domain and isotopic labeling of the C-terminal regulatory PH domain of PDK1. FIG. 3 illustrates isotopic labeling of the N-terminal regulatory PH domain and site-directed spin labeling of the C-terminal kinase domain of PKB2.

In both protein engineering strategies, a soluble PH domain construct is overexpressed and purified from E. coli or other suitable host with uniform isotopic labeling, including, but not limited to, ¹⁵N- and/or ¹³C-isotopic labeling. Next, a series of kinase domain constructs are engineered to contain a single-site cysteine positioned in one of several strategic surface-exposed locations, and each construct is expressed and purified from a suitable host, such as Sf9 insect cells. The single cysteine residue is chemically modified with a small molecule nitroxide paramagnetic spin label.

Next, the isotopic-labeled regulatory domain fragment is chemically joined to the site-directed spin-labeled kinase domain fragment to generate the full-length native protein with a native peptide bond. This ligation reaction can be achieved by any standard molecular biology means, including native chemical ligation reactions, such as intein-mediated protein ligation. Intein-mediated protein ligation involves a chemoselective reaction between a C-terminal thioester and an N-terminal cysteine residue to yield a native peptide bond at the site of ligation. Intein-mediated protein ligation involves engineering an N-terminal cysteine residue on the carboxy-terminal domain. In addition, the amino-terminal domain requires an intein, such as the MxeGyrA intein, to be engineered at the C-terminus. Treatment with a thioreductant, such as 2-mercaptoethanesulfonic acid (MESNA), generates a C-terminal thioester. The N-terminal cysteine of the C-terminal domain reacts with the C-terminal thioester of the N-terminal domain to regenerate the full-length protein kinase with a native peptide bond.

Once the full-length protein kinase is obtained, the chemical shift assignments and NMR solution structure is determined for the regulatory domain, while in its intact position of the native kinase. Then, NMR relaxation studies or MRET is carried out to determine long-range distance restraints between the spin label of the kinase domain and each of the backbone amide protons of the regulatory domain.

During an NMR relaxation experiment, non-equilibrium magnetization is created on each of the isotopic-labeled backbone amide groups, and the rates of decay of magnetization (longitudinal R1 or transverse R2) back to equilibrium are measured for each amide in the regulatory domain in the absence of spin label on the kinase domain (or the presence of the spin label in its reduced diamagnetic form). Then, the magnetic relaxation rate constants (R1 or R2) for each amide group are measured again, with the spin label in its oxidized radical form. The amount that the relaxation rate is increased (ΔR1 or ΔR2) is used to calculate the distance between the spin label and the backbone amide proton according to the Solomon-Blumebergen equations.

The position of the isotopic-labeled domain relative to the site-directed spin-labeled domain can be calculated using a standard distance geometry/simulated annealing protocol, such as CNS(XPLOR). First, the NMR solution structure of the isotopic-labeled regulatory domain construct is chemically connected to the known X-ray structure of the site-directed spin-labeled domain, and only the peptide bonds located in the loop region of the junction between the two domains is allowed to sample different conformations. Distances between the site-directed spin label and the amide protons of the isotopic-labeled domain are given the energy function normally used for NOE restraints in CNS(XPLOR). This is possible since both NOE and paramagnetic distance restraints have an r⁻⁶ distance dependence. Once the position of the isotopic-labeled domain relative to the site-directed spin-labeled domain is determined, the position of the interface between the two domains and any sites of interest, such as clefts or crevices, within or leading to the interface may be determined.

Before carrying out the MRET approach described above, it is necessary to clone, express, and purify the protein kinase of interest and generate and characterize the auto-inhibited forms that are the target of the designed protein kinase modulator. Recombinant expression methods are well known to the skilled artisan and are described, for example, in Sambrook et al., Molecular Cloning: A Laboratory Manual, Cold Springs Harbor Laboratory, 2^{nd} ed., Cold Springs Harbor, N.Y. (1989). Other references describing molecular biology and recombinant DNA techniques include, for example, DNA Cloning 1: Core Techniques, (D. N. Glover, et al., eds., IRL Press, 1995); DNA Cloning 2: Expression Systems, (B. D. Hames, et al., eds., IRL Press, 1995); DNA Cloning 3: A Practical Approach, (D. N. Glover, et al., eds., IRL Press, 1995); DNA Cloning 4: Mammalian Systems, (D. N. Glover, et al., eds., IRL Press, 1995); Oligonucleotide Synthesis (M. J. Gait, ed., IRL Press, 1992); Nucleic Acid Hybridization: A Practical Approach, (S. J. Higgins and B. D. Hames, eds., IRL Press, 1991); Transcription and Translation: A Practical Approach, (S. J. Higgins & B. D. Hames, eds., IRL Press, 1996); R. I. Freshney, Culture of Animal Cells: A Manual of Basic Technique, 4^{th} Edition (Wiley-Liss, 1986); and B. Perbal, A Practical Guide To Molecular Cloning, 2^{nd} Edition; (John Wiley & Sons, 1988); and Current Protocols in Molecular Biology (Ausubel et al., eds., John Wiley & Sons), which is regularly and periodically updated.

Suitable vectors for expression of protein kinase constructs are, for example, bacterial or yeast plasmids, wide host range plasmids and vectors derived from combinations of plasmid and phage or virus DNA. Vectors derived from chromosomal DNA are also included. Furthermore, an origin of replication and/or a dominant selection marker can be present in the vector according to the invention. The vectors according to the invention are suitable for transforming, transfecting, or infecting a host cell. Exemplary plasmid vectors for expression include pFastBac (Invitrogen) for expression of human protein kinases and their kinase domain constructs and pET vectors (Novagen) for expression of regulatory domains.

Protein kinase constructs may be expressed in any cells suitable for use as host cells for recombinant DNA expression, including any eukaryotic or prokaryotic host cells. Thus, a host cell which comprises the DNA or expression vectors according to the invention is also within the scope of the invention. Suitable host cells transformed with the DNA constructs can be fermented and subjected to conditions which facilitate the expression of the heterologous DNA, leading to the formation of large quantities of the desired protein. Non-limiting examples of preferred host cells suitable for protein expression in accordance with the invention include bacterial and Sf9 insect cells.

The recombinant protein kinase domain fragments are purified prior being chemically joined. Selection of an appropriate purification procedure for the chimeric polypeptides present in the host cell extract or culture medium is routine to one skilled in the art, and may be based on the properties of the polypeptides, such a size, charge and function. Methods of purification include centrifugation, electrophoresis, chromatography, dialysis or a combination thereof. As known in the art, electrophoresis may be utilized to separate the proteins in the sample based on size and charge. Electrophoretic procedures are well known to the skilled artisan, and include isoelectric focusing, sodium dodecyl sulfate polyacrylamide gel electrophoresis (SDS-PAGE), agarose gel electrophoresis, and other known methods of electrophoresis.

The purification step may be accomplished by a chromatographic fractionation technique, including size fractionation, fractionation by charge and fractionation by other properties of the polypeptides being separated. As known in the art, chromatographic systems include a stationary phase and a mobile phase, and the separation is based upon the interaction of the polypeptides to be separated with the different phases. In some forms of the invention, column chromatographic procedures may be utilized. Such procedures include partition chromatography, adsorption chromatography, size-exclusion chromatography, ion-exchange chromatography and affinity chromatography. An affinity tag may also be engineered into the desired polypeptide for purification purposes. For example, the DNA constructs of the invention may encode 6-Histidine tags (His₆ tags) to facilitate protein purification on nickel affinity columns.

Reference will now be made to specific examples illustrating the constructs and methods above. It is to be understood that the examples are provided to illustrate preferred embodiments and that no limitation to the scope of the invention is intended thereby.

### EXAMPLES

### Example 1

### Segmental Isotopic Labeling of Either an N-Terminal or C-Terminal Regulatory Domain

To facilitate structural and dynamical studies of yet uncharacterized protein kinases, all general procedures necessary for generating the required soluble and functional domain constructs from the full-length kinases are described. First, soluble and functional “full-length” kinase constructs that contain both the intact contiguous catalytic and regulatory domains of interest are identified. The “full-length” protein kinase is tested for soluble expression in a number of different organisms (e.g., bacteria, yeast, or insect cells). If no soluble expression is observed, then amino acid sequence alignments are performed with sequences of related kinase and regulatory domains from other enzymes and proteins for which three-dimensional structures have been determined. A slightly modified construct may be identified in which a small fragment of the N- and/or C-terminal regions are deleted. Then, sets of primers covering differing ranges of residues are used to generate different constructs to test for soluble expression in the organism of choice. If a construct is identified that contains the contiguous domains of interest, then soluble and functional constructs of the two individual domains can be more easily identified by limited proteolysis, which ultimately facilitates combined segmental isotopic and site-directed spin labeling for NMR studies.

a. Molecular Cloning of Full-Length Protein Kinase Genes from a Human cDNA Library

i. Touchdown PCR Amplification from cDNA Library

The cDNA coding sequence is obtained for the full-length target protein kinase (e.g., PDK1, accession no. NM002613; and PKB2, accession no. NM005163). PCR is used to generate full-length copies of the human cDNA coding sequence. Human tissue Marathon-Ready™ cDNA libraries (Clontech) serve as convenient templates. The Advantage 2 Polymerase Mix (Clontech), which includes TaqStart Antibody for automatic hot start PCR is found to be more effective than other polymerase mixes. In order to further increase the efficiency of gene-specific PCR amplification from the cDNA library, the forward and reverse primers preferably complement the 5′- and 3′-termini of the coding sequence and contain no flanking restriction sites. Each primer should have GC content of 50-70% and a Tₘ≧70° C. In order to increase the specificity of gene amplification, the following optimized protocol for “touchdown” PCR is employed: incubation at 94° C. for 30 s; 5 cycles of 94° C. for 30 s and 72° C. for 3 min; 5 cycles of 94° C. for 30 s and 70° C. for 3 min; and 25 cycles of 94° C. for 30 s and 68° C. for 3 min. The resulting DNA products are efficiently isolated by gel-purification (1% agarose) using the QIAquick® Gel Extraction Kit (Qiagen).

ii. Efficient Ligation and Corrective Mutagenesis

The 3′-dA nucleotide overhangs generated by the Advantage 2 Polymerase Mix (Clontech) are removed by incubation with a blunt-ended pfu-type high-fidelity polymerase mix (e.g., KOD proofreading polymerase, Novagen). The blunt-ended cDNA PCR products are efficiently cloned into the pCR®-Blunt II-TOPO® plasmid vector (Invitrogen), which is supplied linearized with Vaccinia virus DNA topoisomerase I covalently bound to the 3′ end of each DNA strand. The TOPO enzyme catalyzes ligation of the 3′ ends of each vector strand to the 5′ ends of the PCR product, while releasing itself in an energy-conserved reaction. In addition, pCR®-Blunt II-TOPO® allows direct selection of recombinants via disruption of the lethal E. coli gene ccdB permitting growth of only positive recombinants upon transformation.

The products of the ligation reactions are transformed into any number of strains of competent cells of E. coli (e.g., One Shot® TOP10 Chemically Competent E. coli, Invitrogen) and selected colonies are grown in 10 mL of enriched media (e.g., Luria Broth) containing an appropriate antibiotic (e.g., ampicillin for the pCR®-Blunt II-TOPO® plasmid vector). High quality plasmid preparations can be obtained using the QIAprep® Spin Miniprep Kit (Qiagen) for sequence verification.

Plasmids shown to contain the fewest alterations in the cDNA sequence are saved for further corrective mutagenesis using either QuikChange® Single or Multi Site-Directed Mutagenesis Kits (Stratagene) to obtain the native full-length sequences for the target proteins. The newly generated plasmid PCR products are transformed back into competent cells of E. coli, and the plasmids are isolated and sequenced to verify the corrective mutations.

b. Expression and Purification of Human Protein Kinases

i. PCR Subcloning into pFastbac™1 and Generating the Recombinant Bacmid for Producing Recombinant Baculovirus

PCR is used to generate cDNA encoding for an N-terminal His₆ tagged fusion protein of the “full-length” kinase (e.g., residues 51-556 of PDK1 and residues 1-481 of PKB2) containing a PreScission protease recognition sequence for removal of the His₆ tag and flanking restriction enzyme recognition sequences for directional ligation into the pFastbac™1 vector. The full-length protein kinase gene in the pCR®-Blunt II-TOPO® plasmid vector is used as the template to generate the desired coding region. The nucleotide coding region does not contain the restriction enzyme recognition sequences selected for directional ligation, and the amino acid sequence is checked for recognition sites for the protease selected for removal of the His₆ or other affinity tag. The reverse or downstream primer (kinase-R) is designed complementary to the 3′-terminal coding region and extended to include the desired restriction enzyme recognition sequence. Two forward or upstream 5′-primers are used to extend the cDNA protein coding region to include a His₆ tag with a PreScission protease cleavage site and a flanking restriction enzyme recognition sequence. The PreScission-kinase-F1 primer is designed complementary to the kinase coding region and extended in the 5′-direction to include nucleotides coding for the PreScission protease peptide recognition sequence (LEVLFQGP). The His₆-PreScission-F2 primer is designed complementary to the protease peptide recognition sequence (LEVLFQGP) and extended in the 5′-direction to include the N-terminal His₆ tag and the restriction enzyme recognition sequence. The Tₘ values for all of the overlapping regions are temperature optimized for the high-fidelity PCR polymerase mix. Standard PCR reaction conditions using 100 ng of plasmid template, 500 nM of the kinase-R reverse primer, 200 nM of the PreScission-kinase-F1 primer, and 300 nM of the His₆-PreScission-F2 primer yields the full-length cDNA with flanking restriction enzyme cloning sites.

A sequence verified restriction fragment is ligated into the pFastBac™1 vector (Invitrogen), which is used to generate recombinant bacmid for producing recombinant baculovirus using the Bac-to-Bac® Baculovirus Expression System (Invitrogen). The recombinant FastBac™1 plasmid is transformed into DH10Bac™ competent cells of E. coli. When transformed DH10Bac™ cells are grown on LB agar plates containing kanamycin (50 μg/mL), gentamicin (7 μg/mL), tetracycline (10 μg/mL), Bluo-gal (100 μg/mL), and IPTG (40 μg/mL), colonies containing recombinant bacmid are white, while colonies containing unaltered bacmid are blue. The DH10Bac™ cells contain a baculovirus shuttle vector (bacmid) with a mini-attTn7 target site and a helper plasmid. Upon transformation, transposition occurs between the mini-Tn7 element on the recombinant pFastBac™1 vector and the mini-attTn7 target site on the bacmid to generate a recombinant bacmid. The transposition reaction is catalyzed by transposition proteins supplied by the helper plasmid. Insertion of the mini-Tn7 into the mini-attTn7 attachment site on the bacmid disrupts expression of the LacZa peptide.

After selected white colonies are re-streaked, a single isolated large white colony is used to inoculate LB media containing kanamycin (50 μg/mL), gentamicin (7 μg/mL), tetracycline (10 μg/mL), and the high molecular weight recombinant bacmid DNA is isolated using the S.N.A.P.™ MidiPrep Kit (Invitrogen). Since the recombinant bacmid DNA is >135 kb in size, PCR analysis is used to verify the presence of the kinase construct using Taq DNA polymerase High Fidelity, the M13 Forward (−40) and M13 Reverse primers, and the protocol provided by Invitrogen.

ii. Producing Recombinant Baculovirus in SJ9 Insect Cells

One μg of recombinant bacmid and 6 μL of Cellfectin® Reagent are diluted in 200 μL of unsupplemented Grace's Medium, incubated for 45 min at room temperature, and then further diluted with 0.8 mL of unsupplemented Grace's Medium (i.e., no antibiotics). This mixture is then added to individual wells of a 35 mm tissue culture plate that contains 9×10⁵ attached Sf9 cells per well (>97% viability), and the cells are incubated at 27° C. for 5 h.

The bacmid solution is replaced with 2 mL of complete growth media (i.e., Sf-900 II serum-free media with antibiotics), and the cells are incubated at 27° C. for 72 h. The recombinant P1 viral stock is collected as the clarified supernatant after centrifugation of the media containing the cells from which viral budding had been confirmed using an inverted phase microscope at 250-400×. The recombinant P1 viral stock is amplified by infection of a 10 mL suspension culture at 2×10⁶ cells/mL, and the cells are incubated at 27° C. for 48 h.

After centrifugation, the recombinant P2 stock is collected as the clarified supernatant, and a titer of ≧1×10⁷ plaque forming units (pfu)/mL is obtained. Aliquots of the recombinant P2 viral stock are stored either at −80° C. (long term storage) or 4° C. (immediate use).

iii. High Level Protein Kinase Expression in Sf9 Insect Cells

Recombinant P2 viral stocks are used to infect 500 mL spinner flask cultures of Sf9 cells in the mid-logarithmic phase of growth (1.5×10⁶ cells/mL) at a multiplicity of infection (MOI) of 1 yielding 1.5×10⁶/mL. The infected cells are incubated at 27° C. for 72 h, harvested by centrifugation for 10 min at 4° C. at 3000 rpm in a Sorvall centrifuge with a GS-3 rotor, and whole cell pellets are stored at −80° C.

Frozen pellets are allowed to thaw on ice before re-suspending in 20 mL lysis buffer (per 500 mL spinner flask) containing 50 mM Tris-HCl, pH 7.5, 300 mM NaCl, 5 mM EDTA, 1 mM DTT, 1 mM sodium orthovanadate, 5 mM sodium fluoride, 1% (v/v) glycerol, 0.2% (v/v) Triton X-100, and complete protease inhibitor cocktail (one tablet per 50 mL). The cells are lysed by incubation for 20 min, followed by freezing and thawing, and cell debris is pelleted by centrifugation for 30 min at 4° C. at 18,000 rpm in a Sorvall centrifuge with a SS-34 rotor. The supernatants containing the soluble components of the cell lysate are collected.

iv. Nickel Sepharose HisTrap HP Affinity Purification of N-Terminal His₆ Tagged Protein Kinase

The soluble lysate is directly loaded by FPLC (1 mL/min) onto a 5 mL bed volume of Ni Sepharose HisTrap HP affinity column (Amersham) equilibrated at 4° C. in 50 mM Tris-HCl, pH 7.5, 300 mM NaCl, 50 mM imidazole, 5 mM EDTA, 1 mM DTT, and 1% (v/v) glycerol. The column is subsequently washed until the absorbances at 260 nm and 280 nm return to baseline. The recombinant His₆ affinity tagged enzyme is eluted by increasing the imidazole concentration from 50 to 500 mM. Fractions are analyzed by SDS-PAGE, and fractions containing >85% pure recombinant protein are pooled and concentrated. If no soluble protein expression is obtained, then expression of alternative “full-length” constructs is attempted.

v. Characterization of Purified Recombinant Protein Kinases

The identity of the purified enzyme is confirmed by N-terminal Edman sequencing and also by Western blotting if an antibody is available.

The overall molecular weight of the purified kinase is determined by electrospray ionization mass spectrometry (ES-MS) on enzyme preparations that have been either treated or not treated with a variety of protein phosphatases. A reduction in the apparent molecular weight upon treatment with phosphatase indicates that the purified kinase undergoes phosphorylation during protein expression and/or purification. Phosphopeptide mapping studies are carried out to identify the precise sites of phosphorylation and those sites that are dephosphorylated by treatment with specific phosphatases.

The activity of the protein kinase, before and after treatment with specific phosphatases, is evaluated towards model synthetic peptide substrates.

The stability of the purified enzyme is evaluated with regard to ionic strength, temperature, pH, and freeze/thaw cycling. While the enzymatic activity of most human protein kinases, which have been expressed and purified from Sf9 insect cells, is prolonged at lower temperatures (4° C.) in buffers of physiological ionic strength (≧0.15M) and mild pH, NMR protein structural studies are optimally performed at higher temperatures (≧20° C.) in buffers of lower ionic strength (≦0.15M) and slightly acidic pH.

c. Identification of Soluble and Functional Catalytic and Regulatory Domain Constructs for Intein-Mediated Protein Ligation

The “full-length” kinase target construct (20-50 μg) is cleaved at 37° C. with trypsin (0.2-0.5 μg) in 50 mM Tris-HCl, pH 8, with 100 mM NaCl. Aliquots are taken every 1 min over 20 min, supplemented with 10 mM benzamidine, and analyzed by SDS-PAGE. The protein fragments are purified from the gel and subjected to N-terminal Edman sequencing and MALDI-TOF analysis. From knowledge of the N-terminal sequence, the molecular mass, and the trypsin cleavage sites, soluble domain constructs are identified.

After identifying soluble domain constructs, the first approach towards selection of a suitable ligation site is to identify an X-Cys pair between the boundaries of the N- and C-terminal domains to be ligated. X will be the C-terminal residue of N-terminal domain, and Cys will be the N-terminal residue of the C-terminal domain (11,12). If no X-Cys pair (where X is preferably His, Cys, Gly and preferably not Asp, Pro, Ile, or Val) exists between the boundaries, then an X-Ser pair is chosen, since a Ser→Cys mutation is both iso-steric and iso-electronic and often causes very little effects in protein activity and stability. If an X-Ser pair does not exist, then care must be taken to identify a pair of residues for which mutagenesis to form an X-Cys pair is least likely to induce structural perturbations and lower the stability of the enzyme.

d. Generation of N-terminal Cysteine (NT-Cys) on C-Terminal Kinase or Regulatory Domains

Proteolytic cleavage of an N-terminal affinity tag using the Factor Xa protease is the most convenient method for generating an NT-Cys, since Factor Xa cleaves at the C-terminus of arginine in the recognition sequence IEGR (11,12). Although the procedure described below is for removal of an N-terminal His₆ tag, any N-terminal affinity tag may be substituted.

Since a C-terminal larger sized kinase domain does not require isotopic label, high level and soluble protein expression is best achieved in Sf9 insect cells. A fusion protein construct containing an N-terminal His₆ tag with a Factor Xa protease cleavage site prior to an NT-Cys of the kinase domain construct is generated in the FastBac™1 vector (Invitrogen). The recombinant pFastBac™1 vector is used to generate recombinant baculovirus using the Bac-to-Bac® Baculovirus Expression System (Invitrogen), and the His₆ tagged kinase domain is expressed and purified from Sf9 insect cells.

In order to facilitate uniform ¹⁵N and/or ¹³C-isotopic labeling, a C-terminal regulatory domain must be expressed in either bacterial or yeast cells. A protein expression vector containing an N-terminal His₆ tag with a Factor Xa protease cleavage site prior to an NT-Cys of the regulatory domain construct is generated by PCR and transformed into protein expression strains of either E. coli bacteria or P. pastoris yeast. The temperature, time, and chemical inducer (e.g., IPTG) concentration are optimized for high-level expression of a soluble His₆ tagged fusion regulatory domain construct. If no soluble protein can be generated, then alternative ligation sites are considered. If high-level expression of soluble protein is achieved, then uniform ¹⁵N- and/or ¹³C-isotopic labeling is carried out by growing the cells in minimal media containing ¹⁵NH₄Cl as the sole nitrogen source, either ¹³C- or ¹²C-glucose as the sole carbon source, and the appropriate selective antibiotic before His₆ tag affinity purification.

Optimal conditions for Factor Xa proteolytic cleavage of individual affinity tagged fusion proteins are established. Varying amounts of Factor Xa protease are added to the purified His₆ tagged C-terminal domain, and digestion is carried out at varying temperatures for varying times. The extent of cleavage may be followed by HPLC, ES-MS, or SDS-PAGE.

After the reaction is carried out under optimized conditions, Factor Xa is removed by passage over a benzamidine column. The cleaved His₆ tag and any remaining uncleaved protein containing the His₆ tag are removed simultaneously by incubation with nickel sepharose HisTrap HP (200 μL) resin (Amersham). After incubation for 15 min, the mixture is centrifuged and the supernatant containing the cleaved enzyme is concentrated (≧0.05 mM) and stored at −80° C.

e. Generation of C-termtinal Thioester on N-Terminal Kinase and Regulatory Domains

A C-terminal thioester on either an N-terminal kinase domain or N-terminal regulatory domain is generated by designing a construct in which a cleavable N-terminal His₆ tag preceding the domain construct is fused to a C-terminal Mxe GyrA intein (11,12). Depending on whether the N-terminal construct is expressed in bacteria, yeast, or insect cells, such fusion protein constructs are generated by PCR and subsequent ligation into any number of protein expression vectors. Addition of the thiol reagent 2-mercaptoethanesulfonic acid (MESNA) to the purified fusion protein causes cleavage of the C-terminal Mxe GyrA intein and formation of a C-terminal thioester derivative of MESNA with the N-terminal domain. MESNA is particularly advantageous over other thiolytic reagents (e.g., ethanethiol or thiophenol), because it is significantly more soluble and is completely odorless.

Since an N-terminal kinase domain requires expression in Sf9 insect cells, an N-terminal His₆ tag with a PreScission protease cleavage site preceding the kinase domain construct fused to the C-terminal Mxe GyrA intein is generated in the FastBac™1 vector. First, the cDNA encoding for the kinase domain construct with an N-terminal restriction enzyme recognition sequence, His₆ tag, and protease recognition sequence is obtained by PCR using the recombinant pFastBac™1 vector containing the full-length kinase gene as the template. Second, the cDNA encoding for the Mxe GyrA intein is obtained by PCR using the pTWIN1 vector (New England Biolabs) as the template. The Mxe GyrA forward primer is complementary to the N-terminus of the Mxe GyrA intein and extended in the 5′-direction to generate an overlapping region with the C-terminal residues of the kinase domain construct. The Mxe GyrA reverse primer is complementary to the C-terminus of the Mxe GyrA intein and extended in the 5′-direction to include a stop codon and a restriction enzyme recognition sequence. Finally, the cDNA PCR products encoding for the N-terminal kinase domain and the Mxe GyrA intein are joined by further PCR, since both fragments share the nucleotide coding region coding for C-terminus of the kinase domain construct. Then, a sequence verified restriction fragment of the His₆ tagged kinase domain fused at the C-terminus to the Mxe GyrA intein is ligated into the pFastBac™1 vector (Invitrogen).

The recombinant pFastBac™1 vector is used to generate recombinant baculovirus using the Bac-to-Bac® Baculovirus Expression System (Invitrogen), and the Mxe GyrA fusion construct of the kinase domain is expressed in Sf9 insect cells and His₆ tag affinity purified. If no soluble fusion construct can be obtained, then it is necessary to select a new ligation site, and new domain constructs must be engineered.

Since an N-terminal regulatory domain requires uniform ¹⁵N- and/or ¹³C-isotopic labeling by E. coli expression in minimal media, a fusion protein construct containing a cleavable N-terminal His₆ tag preceding the regulatory domain construct fused to a C-terminal Mxe GyrA intein is generated in a chosen bacterial protein expression vector. First, the cDNA encoding for the regulatory domain with an N-terminal cloning recognition sequence is obtained by PCR. Second, the cDNA encoding for the MxeGyrA intein is obtained by PCR and extended in the 5′-direction to generate an overlapping region with the C-terminal residues of the regulatory domain. The cDNA PCR products encoding for the regulatory domain and the Mxe GyrA constructs are joined by further PCR, ligated to a bacterial protein expression vector, and optimized for high-level bacterial expression.

Even if high levels of insoluble protein are produced, it is often possible to obtain the regulatory domain in soluble form after thiolytic cleavage with MESNA of the Mxe GyrA intein under partially denaturing conditions (≦4 M guanidine HCl or urea). Smaller sized regulatory domains can often be refolded to be soluble and functional. Once it has been established that thiolytic cleavage can yield a soluble domain construct (see below), the His₆ tagged regulatory domain-Mxe GyrA construct is expressed in minimal media for isotopic labeling and His₆ tag affinity purified.

The N-terminal His₆ tagged kinase-Mxe GyrA or isotopic-labeled regulatory domain-MxeGyrA construct is subjected to thiolytic cleavage in mild aqueous buffer (pH 6-8) by adding MESNA in a 4-fold excess molar ratio to the protein. The extent of the cleavage reaction may be followed by subjecting small aliquots of the reaction mixture to HPLC, ESMS, or SDS-PAGE analysis. The reaction rate and extent of cleavage is increased under slightly acidic conditions (pH 6-7). The MESNA thioester derivative of the N-terminal domain may be stored under normal protein domain storage conditions until native chemical ligation with the C-terminal domain is ready to be performed.

f. Native Chemical Ligation of Kinase and Regulatory Domains

Native chemical ligation between the N-terminal domain containing the C-terminal thioester derivative of MESNA and the C-terminal domain containing an NT-Cys is typically carried out in mild aqueous buffers (pH 6-8) at temperatures between 4 and 40° C., depending on the stability requirements of the protein components (11,12). The reaction is further catalyzed by including 2% (w/v) MESNA as a “cofactor” in the ligation buffer in the presence of the protein components at the highest possible concentration (≧0.05 mM). The rates of native chemical ligation vary depending on the temperature, protein concentration, and the amino acid residues near the termini being joined. The extent of the ligation reaction may be monitored either by HPLC, ESMS, or SDS-PAGE.

Once it has been established that the reaction has proceeded near completion, the ligated full-length kinase is His₆ tag affinity purified, and the affinity tag is proteolytically removed. The enzymatic properties (e.g., activity, regulation, and stability) of the segmentally-labeled full-length protein kinase are evaluated and compared to those determined for the native full-length protein kinase. In addition, protein NMR spectra are obtained for the segmentally-labeled full-length protein kinase and compared to spectra of the isolated regulatory domain construct.

### Example 2

### Site-Directed Paramagnetic Spin Labeling of Either an N-Terminal or C-Terminal Kinase Domain

a. Generation of Single-Site Cysteine Mutants of Kinase Domains

Alkylation of the thiolate of cysteine residues provides a highly site-specific method for the introduction of spin labels on proteins (13). However, many of the kinase domains contain numerous cysteine residues. If the number of cysteine residues in the kinase domain construct is less than five, then it is practical to use the QuikChange® Single or Multi Site-Directed Mutagenesis Kits (Stratagene) in order to mutate all of the cysteine residues to serine residues. If the kinase domain contains more than five cysteine residues, then it is preferable to generate a cysteine-free mutant by PCR-based gene synthesis of an engineered kinase domain in which all of the cysteines have been mutated to serines (10). With a cysteine-free kinase domain, site-directed mutagenesis can be further used to substitute any amino acid residue with a single cysteine, allowing a spin label to be introduced at any position in the kinase domain.

Using the known X-ray structure of a given kinase domain, four to six residues for cysteine substitution are chosen. Single-site cysteine mutant constructs are generated so that spin label modifications are obtained on both the N- and C-lobes at positions both near and away from the kinase active site. Residues with highly solvent-accessible side chains are preferred, particularly the native cysteine and serine residues. However, substitution of any charged residue can yield a stable mutant.

b. Chemical Modification of Single Cysteine with Nitroxide Spin Label

An N-terminal kinase domain (e.g., PDK1 as depicted in FIG. 2) with a C-terminal thioester derivative of MESNA is directly modified with the spin label reagent (1-oxyl-2,2,5,5-tetramethyl-3-pyrroline-3-methyl)methanesulfonate (MTSL, Toronto Research Chemicals Inc., Canada) (13). The protein is dissolved in the preferred buffer (pH 8), and oxygen is removed by flushing the solution with argon gas. A 3-fold molar excess of MTSL from a 40 mM stock in acetonitrile is added to the protein (0.05-0.5 mM), and the reaction mixture is incubated in the dark at room temperature overnight. The reaction rate may be increased by incubation with higher concentrations of MTSL (≦10-fold molar excess). Excess MTSL reagent is removed by gel filtration on a P4 column.

In order to remove any unlabeled protein with a free sulfhydryl group, the reaction product is passed over an organomercurial thiol-affinity column (Bio-Gel 501, Bio-Rad). A sensitive method for determining the extent of modification is by titration of an aliquot of the reaction product with the thiol-specific fluorophore 7-diethylamino-3-(4′-maleimidylphenyl)-4-methylcoumarin (CPM, Molecular Probes, Eugene, Oreg.). The nitroxide is reduced prior to fluorescence measurements to prevent paramagnetic quenching of the CPM fluorophore. Fluorescence emission spectra are collected using the ratio. The efficiency of spin labeling is evaluated by direct EPR measurements of the spin-labeled protein.

The N-terminal kinase domain containing both a C-terminal thioester and a site-directed nitroxide spin label is chemically ligated to the C-terminal isotopic-labeled regulatory domain.

c. Chemical Protection and Deprotection of N-Terminal Cysteine (NT-Cys) of a C-Terminal Kinase Domain

In the case where a C-terminal kinase domain (e.g., PKB2 as depicted in FIG. 3) requires a single-site cysteine to attach the spin label, but also requires an N-terminal cysteine for intein mediated ligation, the N-terminal cysteine must be chemically protected before chemical modification with the nitroxide spin label. NT-Cys residues are distinguished from internal cysteine residues by the presence of two vicinal nucleophiles, β-thiol and α-amine, which confer unique chemical reactivity with ninhydrin (indane-1,2,3-trione). It is known that ninhydrin reacts with uncharged primary amines (—NH₂) under mild aqueous conditions to form the chromophore Ruhman's purple, which has been greatly utilized for quantitative amino acid analysis. However, the β-thiol of free cysteine or an NT-Cys further reacts with the Schiff's base intermediate of ninhydrin bonded to the N-terminal amino group to form a cyclic 5-membered spirothiazolidine (Thz) ring (14). The Thz structure effectively protects the NT-Cys so that the internal single-site Cys can be chemically modified with the nitroxide spin label. Following attachment of the nitroxide spin label, ninhydrin is removed to facilitate native chemical ligation of the C-terminal site-directed spin labeled kinase domain to the N-terminal regulatory domain. The N-terminal His₆ tag is cleaved with Factor Xa protease to generate the C-terminal kinase domain with an NT-Cys and a single internal Cys.

A 10-fold molar excess of ninhydrin (≦10 mM) is added from a concentrated stock solution to the purified C-terminal kinase domain and incubated in buffer (pH 5-7) at room temperature for 2 h. Excess ninhydrin is removed by gel filtration on a P4 column, and the extent and specificity of the ninhydrin reaction is evaluated. The extent of possible lysine modification is directly assessed by observation of Ruhman's purple color formation. Ninhydrin can be prevented from reacting with lysine by lowering the pH of the protection reaction.

The extent of NT-Cys and internal cysteine modification is assessed by Ellman's reaction. 100 μL aliquots of the test reaction is diluted with 850 μL of phosphate buffer (pH 7.5) and 50 μL of 3 mM 5,5′-dithio-bis(2-nitrobenzoic acid) (DTNB or Ellman's reagent) and the absorbance is measured at 412 nm. Under these conditions, the test reaction should show approximately one half the titratable sulfhydryl groups than an identical protein sample that did not undergo reaction with ninhydrin (corrected for protein concentration). If more than one half sites are obtained, then the ninhydrin is further removed from the internal cysteine by re-passage over the P4 gel filtration column. If less than one half sites are obtained, the ninhydrin reaction is carried out for longer times.

The ninhiydrin protected NT-Cys kinase domain is reacted with MTSL and the extent of modification is evaluated by CPM titration and EPR. Ninhydrin is removed from the NT-Cys of the spin labeled domain by treatment with a 10-fold molar excess of free cysteine at pH 7.5-8.0 for 30 min at room temperature. Free cysteine and the ninhydrin-cysteine compounds are removed by gel filtration, and the extent of the deprotection reaction is evaluated by thiol titration with CPM.

The C-terminal kinase domain containing both an NT-Cys and a site-directed nitroxide spin label is chemically ligated to the N-terminal isotopic labeled regulatory domain.

### Example 3

### NMR Structural Studies

a. NMR Structural Studies of the Regulatory Domain

First, the solution structure of the isotopic-labeled regulatory domain while spliced and intact with its kinase domain is determined by standard heteronuclear multidimensional NMR methods (15). However, the overall rotational correlation times, τ_{c}, of segmental isotopic-labeled full-length kinases will be greater than those of the equivalent isotopic labeled individual regulatory domains, which will cause broadening of the NMR peaks and loss of resolution. By collecting NMR spectra at higher temperatures (e.g., 37° C.), effective rotational correlation times of larger proteins can be reduced, thus enhancing the spectral resolution. If the target full-length protein kinase is not stable at higher temperatures for prolonged data collection times, then NMR peak broadening is reduced by selective incorporation of amino acids with aliphatic side chains containing carbon-bound deuterium instead of hydrogen (e.g., Ala, Val, Leu, and Ile) into the ¹⁵N- and ¹³C-isotopic labeled domain (16). If deuterium labeling is necessary to obtain NMR spectra, then advanced TROSY-modified forms of the NMR experiments described below will yield quality data on higher field NMR instruments (e.g., ≧600 MHz) (17). While this approach provides for enhanced ability to perform heteronuclear multidimensional NMR experiments for assignment of backbone resonances and determination of backbone secondary structure, tertiary structural information is compromised due to the loss of NOE signals that would otherwise indicate hydrophobic packing between side chain methyls from residues distant in residue sequence. In such cases, the structure of the isolated domain construct is determined and used with the X-ray structure of the kinase domain for overall model calculations.

If the isolated regulatory domain construct or the segmentally labeled full-length protein kinase is sufficiently stable in low salt (≦0.15 M) buffer solutions for up to three days, then sequential backbone assignments are initially obtained from ¹⁵N-edited NOESY and TOCSY spectra and confirmed and completed by ¹⁵N- and ¹³C-edited HNCACB and CBCA(CO)NH triple resonance spectra. Side-chain assignments are primarily obtained from ¹⁵N- and ¹³C-edited HCCH—TOCSY, CC(CO)NH, and HC(CO)NH experiments.

Intramolecular distance constraints between protons of the regulatory domain are obtained from ¹⁵N- and ¹³C-edited NOESY spectra. NOE signals to specific residues of the ¹³C-isotopically labeled regulatory domain from protons of the unlabeled kinase domain are distinguished by performing a ¹²C/¹³C-isotope edited NOESY experiment, which detects NOEs to methyl protons on ¹³C-labeled residues only from ¹²C-bound protons. Dihedral angle restraints are determined from ³J_{NHα} values calculated from HNCA-J and HNHA experiments.

If an adequate number of NOE signals between amino acid residues distant in primary sequence (long-range NOE) are determined, structures of the isolated or segmentally-labeled regulatory domain are calculated from randomized initial structures using the hybrid distance geometry-simulated annealing and protocol in the program CNS(XPLOR) (18). A set of substructures are selected to undergo a simulated annealing refinement to select for a final ensemble of energy-minimized structures that satisfy the criteria of no NOE violations of >0.5 and no dihedral violations of >5° to be used to define the tertiary structures of the regulatory domain construct.

In order to determine the position of the regulatory domain with respect to the kinase domain, it is necessary to confirm or re-determine as many chemical shift assignments as possible for regulatory domain backbone amide cross peaks in two-dimensional ¹H-¹⁵N HSQC spectra of the full-length kinase. Backbone amide chemical shift assignments of the intact regulatory domain facilitate the NMR relaxation experiments combining segmental isotopic and site-directed spin labeling.

b. Distance Restraints between Backbone Amides of Regulatory Domain and Site-Directed Spin Label of Kinase Domain

Distances between the unpaired electron of the nitroxide spin label on the kinase domain and each of the backbone amide protons of the ¹⁵N-labeled regulatory domain are calculated from the amount that either the longitudinal (ΔR1, Eq. 1) or transverse (ΔR2, Eq. 2) relaxation rates of the amide protons are increased in the presence of the spin label according to a modified form of the Solomon-Bloembergen equation (13,19):

$$\begin{matrix}r = \sqrt[6]{\frac{2 K}{Δ R 1} \times \frac{3 {τ}_{c}}{1 + {ω}_{H}^{2} {τ}_{c}^{2}}} & ( 1 ) \\ r = \sqrt[6]{\frac{K}{Δ R 2} \times [ 4 {τ}_{c} + \frac{3 {τ}_{c}}{1 + {ω}_{H}^{2} {τ}_{c}^{2}} ]} & ( 2 )\end{matrix}$$

where K is a constant (1.23×10⁻³² cm⁶ s⁻²) for paramagnetic nitroxide, ωₕ is the Larmor frequency of the amide proton (sec⁻¹), τ_{c} is the correlation time for the electron-amide proton vector (sec), and r is the vector distance between the electron and the proton (cm). The paramagnetic enhancement of the longitudinal (ΔR1) and transverse (ΔR2) relaxation rates are calculated from Eqs. 3 and 4:
1/T1ₚ=ΔR1=R1ₚₐᵣₐ−R1_{dia} (3)
1/T1ₚ=ΔR2=R2ₚₐᵣₐ−R2_{dia} (4)
where R1ₚₐᵣₐ and R2ₚₐᵣₐ are the longitudinal and transverse relaxation rates of the amide proton in the presence of oxidized paramagnetic nitroxide, and R1_{dia} and R2_{dia} are the relaxation rates of the amide proton in the presence of reduced diamagnetic nitroxide or no spin label at all.

With good approximations of τ_{c} and paramagnetic relaxation enhancement values for either ΔR1 or ΔR2, long range distance restraints for electron-amide proton vectors are obtained from Eqs. 1 and 2, which can be used to evaluate the overall structural dynamics of the contiguous regulatory and kinase domains. Although the distance restraints obtained from only one site-directed spin label will give good indication of the overall arrangement between the regulatory and kinase domains, additional distance restraints obtained from NMR studies of other engineered constructs in which the spin label is positioned at different locations on the kinase domain will give better indication of the overall structural dynamics of the full-length protein kinase.

i. Distance Restraints Derived from either ΔR1 or ΔR2

Longitudinal R1 and transverse R2 relaxation rates of the backbone amide protons of an ¹⁵N-labeled regulatory domain are measured using standard ¹H-¹⁵N HSQC pulse sequences, which have been modified to include either an inversion recovery sequence (R1) or a CPMG phase-cycled spin echo (R2) (20,21). Inversion recovery HSQC spectra are collected with varying recovery delay times (e.g., 0, 25, 75, 150, 300, 500, 700, 1000, 1500, 2000, 2500, and 3000 ms); and CPMG HSQC spectra are collected with a constant spin echo delay of 0.5 ms and with varying echo trains (e.g., 0, 5, 10, 20, 30, 50, 75, 100, and 150 echos).

Integrated peak volumes (V) are measured for each amide cross peak in the two-dimensional spectra and plotted as a function of either the inversion recovery delay time for R1 measurements or the time in the transverse plane (number of echo trains×constant echo delay of 0.5 ms) for R2 measurements. In order to account for the small decrease in peak volume that occurs during the HSQC pulse sequence, it is preferable if the peak volumes are fitted to Eq. 5 (22):
V(τ)=V_{D}[1−B(1−exp(−κR))×exp(−τR)] (5)
where V_{D} is the initial peak volume, κ is the sum of acquisition and preparation times during the ¹H-¹⁵N HSQC experimental pulse sequence, B is an adjustment parameter for incomplete magnetization inversion, and R can be either the longitudinal (R1) or transverse (R2) relaxation rate constant.

Longitudinal R1 or transverse R2 relaxation rates are determined for the amide proton relaxation rates under both diamagnetic and paramagnetic conditions, and the paramagnetic enhancements (ΔR1 or ΔR2) are calculated by either Eq. 3 or 4, respectively. Traditionally, relaxation spectra are first collected with the spin label in its paramagnetic or oxidized form; identical data is then collected on the same sample in which the spin label has been reduced to its diamagnetic form. To reduce the nitroxide free radical to its secondary amine, a 3-fold molar excess of ascorbate is added and the sample is allowed to incubate at pH 5.3 and room temperature overnight (13). The pH must be readjusted before collecting the HSQC on the reduced sample. If the protein sample is unstable towards treatment with ascorbate, it is possible to determine the R1_{dia} or R2_{dia} values on a segmentally-labeled protein kinase, which has not been chemically modified with the spin label and under identical solution conditions (e.g., buffer, temperature, and protein concentration) as the samples that contain a spin label. Then, the R1_{dia} and/or R2_{dia} values can be subtracted from R1ₚₐᵣₐ and/or R2ₚₐᵣₐ values determined for all of the different single cysteine site-directed spin labeled constructs, and values of ΔR1 and/or ΔR2 may then be used to calculate distances for each of the electron-amide proton vectors according to Eq. 1 and/or 2, respectively.

ii. Rapid Determination of ΔR2 for Unstable Protein Kinases

The primary drawback of determining values of ΔR1 and ΔR2 is that successive HSQC experiments must be carried out with varying inversion recovery or CPMG periods. As these periods become longer, the data collection times of the experiments becomes longer. With minimal sample concentrations (˜0.3 mM), many transients must be collected, and a complete R1 or R2 data set may require days in order to obtain good signal to noise ratios (S/N≧10), which ultimately reduces propagated errors in distance calculations. This can become a hindrance when extensive NMR time is not readily available or when the stability of the engineered protein kinase construct is in question. In such cases, it is possible to simply collect a standard HSQC spectrum of the protein kinase in the presence (paramagnetic conditions) and absence (diamagnetic conditions) of the spin label. Values of the paramagnetic relaxation enhancement effect on the transverse relaxation rate (ΔR2) for each amide proton are determined from the ratio of the intensity (height) of the HSQC cross peak in the paramagnetic sample (Iₚₐᵣₐ) to the intensity of HSQC cross peak in the diamagnetic sample (I_{dia}) according to Eq. 6 (23,24):

$$\frac{{I}_{para}}{{I}_{dia}} = \frac{R {2}_{dia} exp ( - Δ R 2 t )}{R {2}_{dia} + Δ R 2} \qquad ( 6 )$$

where t is the duration of the INEPT delays (˜9-10 ms) in the HSQC pulse sequence and R2_{dia} is calculated from the line width at half height (R2_{dia}=π×LW) of the amide cross peak in the proton dimension. Values of ΔR2 are obtained by computer fitting of Iₚₐᵣₐ/I_{dia} to Eq. 6 with substitution of known values of R2_{dia} and t. These ΔR2 values are substituted into equation 2 to obtain distances. Since intensity ratios (Iₚₐᵣₐ/I_{dia}) are used to calculate ΔR2, it is required that the two HSQC spectra are collected and processed with identical parameters and that the sample conditions are also identical, especially protein concentration.
c. Calculation of Electron-Amide Proton Vector Correlation Times, τ_{c}

The correlation time τ_{c} required for calculating distances in Eqs. 1 and 2 is described by the sum of contributions from the relaxation of the electron plus motions of the electron-proton vector according to Eq. 7 (13):

$$\frac{1}{{τ}_{c}} = \frac{1}{{τ}_{S}} + \frac{1}{{τ}_{R}} \qquad ( 7 )$$

where τ_{S} is the longitudinal relaxation time of the nitroxide free radical (≧100 ns) and τ_{R} is the effective rotational correlation time of the vector (˜1-30 ns). Since the effective rotational correlation times of the electron-amide proton vectors (τ_{R}) in protein kinase constructs containing a single catalytic kinase domain (˜35-50 kDa) and a small regulatory domain (˜10-20 kDa) will be always be significantly shorter than the longitudinal relaxation time of the nitroxide free radical (τ_{S}), the value of τ_{c} can be approximated from measurements of τ_{R}. Since the distance r depends on the sixth root of τ_{c}, distance calculations will be relatively insensitive to errors in estimate values of τ_{c}. Thus, typical errors of ±10% in ΔR and ±50% in τ_{c} measurements result in an error of only ±8% in the distance.

i. Estimate of τ_{c}: NMR Measurements of ΔR1 at Two Field Strengths

The correlation times, τ_{c}, for each of the individual electron-amide proton vectors may be obtained by measuring ΔR1 at two different magnetic field strengths. For example, τ_{c} for each of the electron-amide proton vectors is calculated from the frequency dependence of the paramagnetic effects at 500 MHz and 700 MHz according to Eq. 8 (19):

$${τ}_{c} = \sqrt[.]{\frac{T {1}_{p 700} - T {1}_{p 500}}{T {1}_{p 500} {ω}_{500}^{2} - T {1}_{p 500} {ω}_{700}^{2}}} \qquad ( 8 )$$

where ω is the larmor frequency of the proton and T1ₚ=1/ΔR1 either at 500 or 700 MHz.

ii. Estimate of τ_{c}: NMR Measurements of ΔR1 and ΔR2 at One Field Strength

The correlation times, τ_{c}, for each of the individual electron-amide proton vectors may also be obtained by measuring both ΔR1 and ΔR2 at one magnetic field strength. For example, τ_{c} for each of the electron-amide proton vectors is calculated from the ratio of ΔR2 to ΔR1 using Eq. 9 (13):

$${τ}_{c} = \sqrt{\frac{6 ( Δ R 2 / Δ R 1 ) - 7}{4 {ω}_{H}^{2}}} \qquad ( 9 )$$

where ω is the larmor frequency of the proton.

iii. Estimate of τ_{c}: EPR Measurement and Spectral Simulation

The electronic rotational correlation time of the nitroxide unpaired electron can be measured by generating simulated EPR spectral lines that reproduce the spectral lines observed from direct EPR studies of the spin labeled protein (19). Spectral simulations can be performed with the available software provided by the EPR manufacturer, and this τ_{c} can be used for calculating the distance for each individual electron-amide proton vector. Although distances calculated in this manner have a larger uncertainty than those calculated using individual correlation times, it can save considerable time and provide reliable overall structures.

d. Structural Calculations

The changes in the position of the isotopically-labeled domain relative to the site-directed spin-labeled domain are calculated using distance geometry/simulated annealing protocols in CNS(XPLOR) (19,24,25). First, the NMR solution structure of the isotopically-labeled domain construct is chemically connected to the known X-ray structure of the site-directed spin-labeled domain, and only the peptide bonds located in the loop region of the junction between the two domains are allowed to sample different conformations. Distances between the site-specific spin label and the amide protons of the ¹⁵N-labeled domain are given the energy function normally used for NOE restraints in CNS(XPLOR). This is possible since both NOE and paramagnetic distance restraints have an r⁻⁶ distance dependence. For each construct, lower and upper bounds for distance restraints are initially derived by propagation of errors for taking the mathematical difference between relaxation rate constants measured under paramagnetic (Rₚₐᵣₐ) and diamagnetic (R_{dia}) conditions, as well as the estimated error in τ_{c}. The most accurate protocol is to generate an annealing process using distance restraints from the backbone amides to the site-directed spin label. In such a case, the amino acid site in the X-ray structure is replaced with the disulfide linked nitroxide molecule and this modification is parameterized for computer simulated annealing calculations. Due to considerable error in distance calculations, much time can be saved if the terminal heteroatom of the native amino acid is used for distance geometry location point of the nitroxide ion, while simply increasing the upper bound of the distance restraints.

The observation that no suitable crystals have been obtained for X-ray diffraction studies of any full-length serine-threonine protein kinase with a regulatory domain strongly suggests that multiple tertiary arrangements may exist between the two domains. Therefore, it is unreasonable to quantify a degree of resolution for such dynamical structures. For example, the sixth-power relationship between r and ΔR will cause the calculated distances to be heavily biased in favor of the shortest distances attained by the regulatory domain amides to the spin label. While this is not a concern for a well-defined rigid domain-domain interaction, it can be misleading for highly flexible domains that sample a wide variety of relative positioning. Nevertheless, the dynamical range of relative orientations between two domains can best be observed by performing distance restraint calculations for numerous additional individual constructs containing the nitroxide spin label on four opposite faces of the kinase. By comparing structures derived for each of the individual constructs, the relative degree of domain-domain flexibility is determined. If the relative positions of the two domains are very similar for all of the spin-labeled constructs, then structure calculations can be carried out using all of the distance restraints.

While the invention has been illustrated and described in detail in the drawings and foregoing description, the same is to be considered as illustrative and not restrictive in character, it being understood that only the preferred embodiments have been shown and described and that all changes and modifications that come within the spirit of the invention are desired to be protected. In addition, all references cited herein are indicative of the level of skill in the art and are hereby incorporated by reference in their entirety.

### REFERENCES

1. Cohen, P. (2002) Protein kinases—the major drug targets of the twenty-first century? Nat. Rev. Drug Discov. 1, 309-315.

2. Noble, M. E. M., Endicott, J. A., and Johnson, L. N. (2004) Protein kinase inhibitors: insights into drug design from structure. Science 303, 1800-1805.

3. Cohen, P. (1999) The development and therapeutic potential of protein kinase inhibitors. Curr. Opin. Cheni. Biol. 3, 459-465.

4. Davies, S. P., Reddy, H., Caivano, M., and Cohen, P. (2000) Specificity and mechanism of action of some commonly used protein kinase inhibitors. Biochem. J. 351, 95-105.

5. Schindler, T., Bornmann, W., Pellicena, P., Miller, W. T., Clarkson, B., and Kuriyan, J. (2000) Structural mechanism of STI-571 inhibition of Abelson tyrosine kinase. Science 289, 1938-1942.

6. Jencks, W. P. (1981) On the Attribution and additivity of binding energies. Proc. Natl. Acad. Sci USA 78, 4046-4050.

7. Erlanson, D. A., Wells, J. A., and Braisted, A. C. (2004) Tethering: fragment-based drug discovery. Annu. Rev. Biomol. Struct. 33, 199-223.

8. Mountain, V. (2003) Astex, Structural Genomix, and Syrrx, I can see clearly now: structural biology and drug discovery. Chem. and Biol. 10, 95-98.

9. Harris, T. K. (2003) PDK1 and PKB/Akt: ideal targets for developing new strategies to structure-based drug design. IUBMB Life 55, 117-126.

10. Gao, X., Yo, P., Keith, A., Ragan, T. J., and Harris, T. K. (2003) Thermodynamically balanced inside-out (TBIO) PCR-based gene synthesis: a novel method of primer design for high-fidelity assembly of longer gene sequences. Nuc. Acids Res. 31, e143.

11. Blaschke, U. K., Silberstein, J., and Muir, T. W. (2000) Protein engineering by expressed protein ligation. Methods Enzymol. 328, 478-496.

12. Cowburn, D. and Muir, T. W. (2001) Segmental isotopic labeling using expressed protein ligation. Meth. Enzymol. 339,41-54.

13. Kosen, P. A. (1989) Spin labeling of proteins. Methods Enzymol. 177, 86-121.

14. Pool, C. T., Boyd, J. G., and Tam, J. P. (2004) Ninhydrin as a reversible protecting group of amino-terminal cysteine. J. Peptide Res. 63, 223-234.

15. Cavanaugh, J., Fairbrother, W. J., Palmer, A. G., and Skelton, N. J. (1996) Protein NMR Spectroscopy: Principles and Practice. Academic Press, Inc., San Diego, Calif.

16. Goto, N. K. and Kay, L. E. (2000) New developments in isotope labeling strategies for protein solution NMR spectroscopy. Curr. Opin. Struct. Biol. 10, 585-592.

17. Fernandez, C. and Wider, G. (2003) TROSY in NMR studies of the structure and function of large biological molecules. Curr. Opin. Struct. Biol. 13, 570-580.

18. Brüinger, A. T. (1992) X-PLOR (Version 3.1), A Systemfor X-ray Crystallography and NMR. Yale University Press, New Haven, Conn.

19. Gaponenko, V., Howarth, J. W., Columbus, L., Gasmi-Seabrook, G., Yuan, J., Hubbell, W. L., and Rosevear, P. R. (2000) Protein global fold determination using site-directed spin and isotope labeling. Protein Sci. 9, 302-309.

20. Carr, H. Y. and Purcell, E. M. (1954) Effects of diffusion on free precession in nuclear magnetic resonance experiments. Phys. Rev. 94, 630-641.

21. Meiboom, S. and Gill, D. (1958) Modified spin-echo method for measuring nuclear relaxation times. Rev. Sci. Instrum. 91, 688-691.

22. Ferretti, J. A. and Weiss, G. H. (1989) One dimensional Overhauser effects and peak intensity measurements. Methods Enzymol. 176, 3-11.

23. Gillespie, J. R. and Shortle, D. (1997) Characterization of long-range structure in the denatured state of Staphylococcal nuclease. I. Paramagnetic relaxation enhancement by nitroxide spin labels. J. Mol. Biol. 268, 158-169.

24. Battiste, J. L. and Wagner, G. (2000) Utilization of site-directed spin labeling and high resolution heteronuclear nuclear magnetic resonance for global fold determination of large proteins with limited nuclear Overhauser effect data. Biochemistry 39, 5355-5365.

25. Gillespie, J. R. and Shortle, D. (1997) Characterization of long-range structure in the denatured state of Staphylococcal nuclease. II. Distant restraints from paramagnetic relaxation and calculation of an ensemble of structures. J. Mol. Biol. 268, 170-184.

We claim:

1. A method of designing a protein kinase modulator to interact at the interface between a first domain and a second domain of a protein kinase, comprising determining the position of at least one site between the first domain and the second domain, wherein the modulator binds to the at least one site and wherein the at least one site is not an ATP binding site.

2. The method of claim 1, wherein the modulator stabilizes an inactive form of the kinase.

3. The method of claim 1, wherein the first domain is site-directed spin-labeled and the second domain is isotopically-labeled.

4. The method of claim 1, wherein the first domain is isotopically-labeled and the second domain is site-directed spin-labeled.

5. The method of claim 1, wherein the first domain and the second domain are chemically ligated.

6. The method of claim 1, wherein magnetic resonance energy transfer studies are used to determine the position of the at least one site between the first domain and the second domain.

7. The method of claim 1, wherein the position of the at least one site is determined by obtaining the distance between the spin label on the first domain to each of the backbone amide protons of the isotopically-labeled second domain.

8. The method of claim 1, wherein the modulator stabilizes an inactive form of the kinase in an allosteric manner.

9. The method of claim 1, wherein the first domain and the second domain are contiguous.

10. The method of claim 1, wherein the first domain is a regulatory domain and the second domain is a catalytic domain.

11. The method of claim 1, wherein the first domain is a catalytic domain and the second domain is a regulatory domain.

12. The method of claim 1, wherein the protein kinase is selected from the group consisting of PDK1; PKB2; PKA; JNK/p38; ERK1,2; CDK; and CHK.

13. The method of claim 10 or claim 11, wherein the regulatory domain is a PH domain.

14. The method of claim 1, wherein the at least one site is at an interfacial cleft or crevice between the first domain and the second domain.

15. The method of claim 1, wherein the position of at least two sites between the first domain and the second domain is determined and the modulator is designed to bind to the at least two sites.

16. The method of claim 1, wherein the modulator comprises at least one small molecule fragment.

17. The method of claim 16, wherein the fragment binds to the at least one site between the first domain and the second domain.

18. The method of claim 1, wherein the modulator comprises at least two small molecule fragments.

19. The method of claim 18, wherein each fragment binds to at least one site between the first domain and the second domain.

20. The method of claim 18, wherein at least one fragment binds to at least one site between the first domain and the second domain and at least one fragment binds to at least one site on the first domain.

21. The method of claim 18, wherein at least one fragment binds to at least one site between the first domain and the second domain and at least one fragment binds to at least one site on the second domain.

22. The method of any one of claims 19-21, wherein the fragments are chemically linked.

23. The method of any one of claims 19-21, wherein the fragments are disulfide-linked.

24. The method of claim 1, wherein the modulator further binds to at least one site on the first domain.

25. The method of claim 1, wherein the modulator further binds to at least one site on the second domain.

26. A method of designing a protein kinase modulator to interact at the interface between a first domain and a second domain of a protein kinase, comprising identifying at least one small molecule fragment that binds to at least one site between the first domain and the second domain, wherein the at least one site is not an ATP binding site.

27. The method of claim 26, further comprising identifying a second small molecule fragment that binds to at least a second site between the first domain and the second domain; and chemically linking the two fragments, wherein the first site and second site are not ATP binding sites.

28. The method of claim 26 or claim 27, wherein the modulator stabilizes an inactive form of the kinase in an allosteric manner.

29. The method of claim 26 or claim 27, wherein the first domain and the second domain are contiguous.

30. The method of claim 26 or claim 27, wherein the first domain is a regulatory domain and the second domain is a catalytic domain.

31. The method of claim 26 or claim 27, wherein the first domain is a catalytic domain and the second domain is a regulatory domain.

32. The method of claim 26 or claim 27, wherein the at least one first site is at an interfacial cleft or crevice between the first domain and the second domain.

33. The method of claim 26, wherein the modulator further comprises at least a second small molecule fragment that binds to at least a second site between the first domain and the second domain.

34. The method of claim 26, wherein the modulator further comprises at least a second small molecule fragment that binds to at least one site on the first domain.

35. The method of claim 26, wherein the modulator further comprises at least a second small molecule fragment that binds to at least one site on the second domain.

36. A method of modulating a protein kinase using a protein kinase modulator designed to interact at the interface between a first domain and a second domain of a protein kinase, wherein the modulator binds to the at least one site between the first domain and the second domain and wherein the at least one site is not an ATP binding site.

37. The method of claim 36, wherein the modulator stabilizes an inactive form of the kinase in an allosteric manner.

38. The method of claim 36, wherein the protein kinase is selected from the group consisting of PDK1; PKB2; PKA; JNK/p38; ERK1,2; CDK; and CHK.

39. The method of claim 36, wherein the at least one first site is at an interfacial cleft or crevice between the first domain and the second domain.

40. The method of claim 36, wherein the position of at least two sites between the first domain and the second domain is determined and the modulator is designed to bind to the at least two sites.

41. The method of claim 36, wherein the modulator comprises at least one small molecule fragment.

42. The method of claim 41, wherein the fragment binds to the at least one site between the first domain and the second domain.

43. The protein kinase modulator designed by the method of claim 1.

44. The protein kinase modulator designed by the method of claim 26.
